(19) United States
(12) Patent Application Publication
(10) Pub. No.: US 2003/0176924 A1
(43) Pub. Date: Sep. 18, 2003

(54) Spinal implant

(76) Inventor: Christopher Burdett, West Palm Beach, FL (US)

Correspondence Address: MCHALE & SLAVIN, P.A., 2855 PGA BLVD, PALM BEACH GARDENS, FL, 33410, US

(21) Appl. No.: 10/097,671

(22) Filed: Mar. 15, 2002

(51) Int. Cl. A61F 2/44

(52) U.S. Cl. 623/17.16

(57) ABSTRACT

A modular spinal implant for stabilizing the spine is inserted between adjacent vertebrae after the removal of the intervertebral disk. The implant has a main body that may be connected with one of several different sized inserts to maintain or attain a desired spinal orientation. The insert and main body are connected by a fin received within a slot in the main body.

### FIELD OF THE INVENTION

This invention relates to the field of orthopedic and neuro-surgery and, more particularly, to implants to be placed between vertebrae in the spine.

### BACKGROUND OF THE INVENTION

Spinal stabilization is one approach to alleviating chronic back pain caused by displaced disk material or excessive movement of individual vertebrae. Conventional stabilization techniques include fusing two or more vertebrae together to circumvent or immobilize the area of excessive movement. Normally, the vertebral disk material which separates the vertebrae is removed and bone graft material is inserted in the space for interbody fusion. In addition to or, in place of, the bone graft material, a spinal implant may be inserted in the intervertebral space.

The conventional surgical approach for stabilization has been posteriorly for ease of access to the spine and to avoid interfering with internal organs and tissue. As the art of spinal implants has expanded, different implant design has allowed or dictated other surgical approaches for proper placement of the implant and correct stabilization of the spine.

### DESCRIPTION OF THE PRIOR ART

U.S. Pat. No. 6,120,506 issued Sep. 19, 2000 to Kohrs et al discloses a lordotic implant and a tap for use in preparing the vertebrae. The implant is designed to be inserted between the non-parallel end plates of adjacent vertebrae and maintain the natural lordotic angle of the spine. This is done through the use of a threaded tapered plug inserted in a tapped hole in the direction required by the lordosis of the spine. The implant is hollow and has radial apertures for accommodating bone graft material.

U.S. Pat. No. 6,015,436 issued Jan. 18, 2000 to Shoenhoeffer discloses a tubular spinal implant. The implant is hollow and has radial apertures for interbody fusion through bone growth material. The device is placed between adjacent vertebrae with the opposite ends of the tube contacting the opposing vertebrae. The opposite ends are threaded together to form the hollow tube.

### SUMMARY OF THE INVENTION

A modular spinal implant for stabilizing the spine by insertion between adjacent vertebrae after the removal of the intervertebral disk. The implant has a main body that may be connected with one of several different sized inserts to maintain or attain a desired spinal orientation. The main body is inserted between adjacent vertebrae with the fixed insert extending anteriorly therefrom for engaging the opposing surfaces of adjacent vertebrae.

Accordingly, it is an objective of the instant invention to teach a lateral anterior surgical approach for placement of a particularly shaped spinal implant for interbody fusion.

It is a further objective of the instant invention to teach a spinal implant having lateral and anterior support for adjacent vertebrae.

It is yet another objective of the instant invention to teach an implant facilitating interbody fusion through bone graft or an ingrowth-type implant, such as bone inductive material.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 shows an exploded view of the implant of this invention;

FIG. 2 is a perspective of an alternate construction of the implant of this invention;

FIG. 3 is a side view of the implant in position within a spine;

FIG. 4 is a superior view, partly in section, of the implant in position; and

FIG. 5 is a side view of the implant showing apertures.

### DETAILED DESCRIPTION OF THE INVENTION

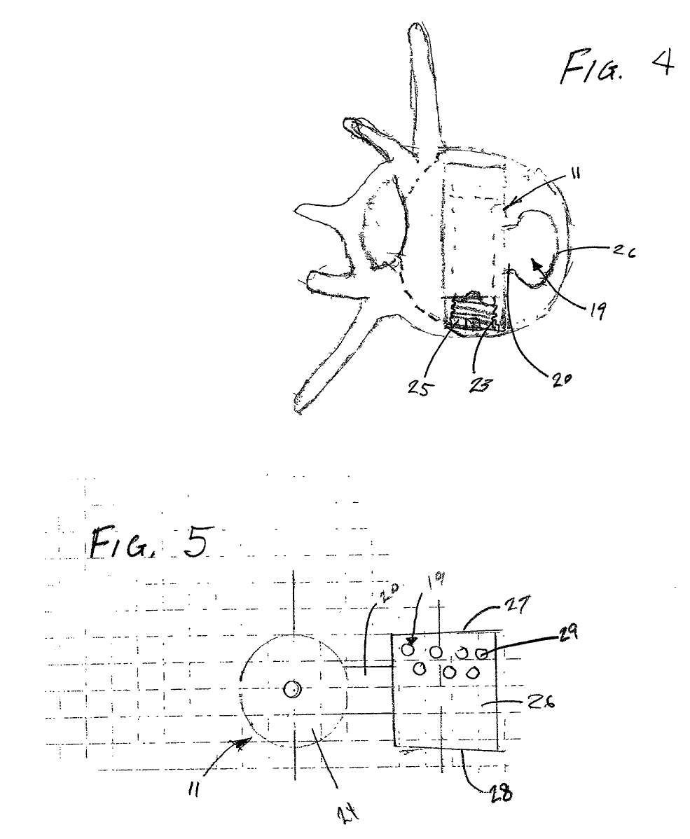

The spinal implant 10 may be placed between two adjacent vertebrae, labeled L-4 and L-5, in FIG. 3, approaching the spine surgically by an lateral approach. After the vertebrae have been visualized, the disk material between the vertebrae is removed and the opposing vertebral surfaces prepared by reaming and sizing. This surgical procedure would also include the alignment or orientation of the opposing surfaces to relieve stress and/or pain. A spinal implant would be chosen from a selection of sizes to best accommodate the intervertebral space of the patient. In so choosing the implant, several main bodies may be paired with several inserts until the best combination is achieved for each patient.

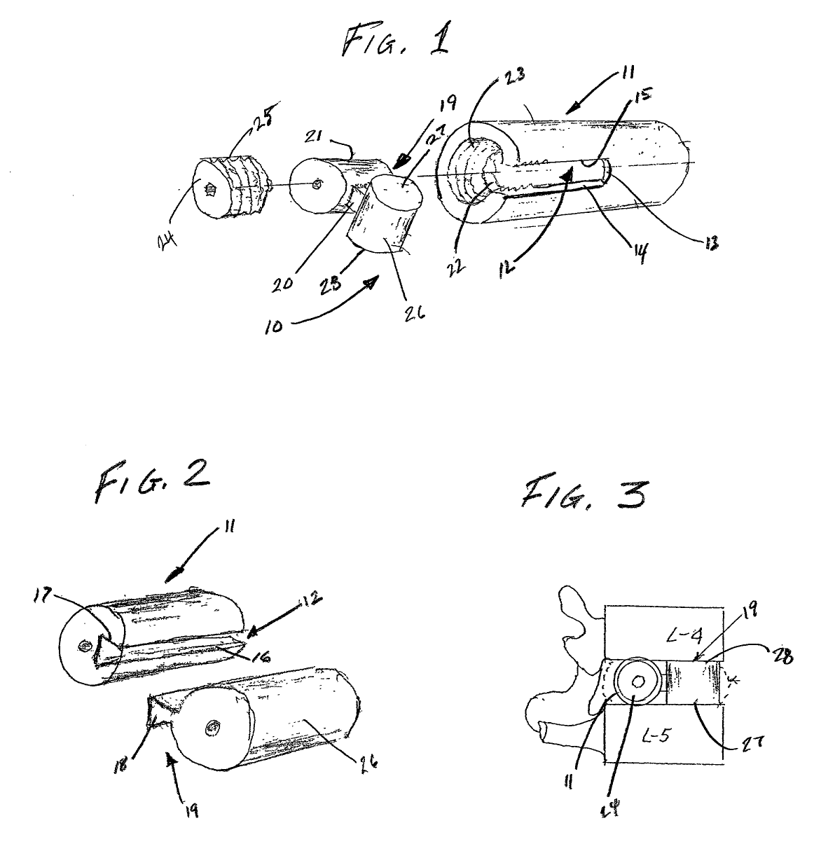

The main body 11, shown in FIG. 1, is cylindrical however, it may be of other shapes. Rather than cylindrical, the main body may be shaped as a rod or a solid configuration, as shown in FIG. 2. The rounded outer shape is preferred in the event of some minor movement of the vertebrae. The main body has a length approximating the width of the vertebrae, as shown in FIG. 3. The wall of the main body has a slot 12 which extends from one end toward the other end. The slot terminates in and end wall 13. The edges 14 and 15 of the slot may be parallel to each other. As shown in FIG. 2, the edges 16 and 17 of the slot are sloped to define a keystone cross section. The keystone slot is used with a keystone shaped fin 18. Also shown in FIG. 2, the slot 12 extends throughout the length of the main body 11. The keystone shaped slot and fin cooperate to form a simple interlocked fit which cannot separate.

In FIG. 1, the insert 19 has an integral fin 20 attached to an interlock 21. The interlock 21 is sized to the internal diameter of the main body 11 and the fin 20 has a thickness similar to the width of the slot 12. One end of the main body has an aperture 22 which has internal threads 23. A closure plug 24 has external threads 25 to engage the internal threads 23. After selection of a suitable insert, the interlock is slid into the main body with the fin extending through the slot. The closure is then threaded into the aperture with the closure plug forcing the interlock and fin against the end wall of the main body. This forms a rigidly coupled modular unit of appropriate size.

As shown in FIG. 4, the insert has a rounded anterior wall 26 approximating the shape of the intervertebral space. The rounded anterior wall joins opposing planar faces 27 and 28 enclosing a space with a volume similar to the size of the intervertebral disk material. As shown in FIG. 5, the anterior wall may have small apertures 29 therethrough. The space my be filled with bone growth material, including bone grafts, to accelerate the fusion of the implant to the spine by bony ingrowth. The anterior wall and the wall of the main body may be provided with a surface treatment to promote bony ingrowth in addition or as an alternative to the apertures. This treatment could be in the form of a roughened surface and/or coatings of compositions enhancing bone growth.

As shown in FIG. 2, the insert 19 has a similar shape to the main body 11. The anterior wall 26 has a surface to facilitate bony ingrowth. The keystone fit between the fin and the slot may be a friction fit preventing lateral dislocation. In this regard, the slot and the fin may have a cooperating slope along their respective lengths requiring increasing pressure as they are telescoped together.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

What is claimed is:

1. A spinal implant for stabilizing a spine comprising a main body adapted for insertion between adjacent vertebrae approximately perpendicular to the longitudinal axis of a spine, said main body having a fixed insert extending anteriorly therefrom for engaging the opposing surfaces of the adjacent vertebrae, said insert removably connected to said main body.

2. A spinal implant of claim 1 wherein said main body has an elongated wall with a left end and a right end, said elongated wall having a slot, said insert having a fin, said fin removably interlocked in said slot.

3. A spinal implant of claim 2 wherein one end of said main body has an aperture with a closure, said slot extending from said aperture toward the other end, said fin slidably connected in said slot, said fin retained in said slot by said closure.

4. A spinal implant of claim 1 wherein said anteriorly extending insert has planar opposing faces adapted to contact adjacent vertebrae, said opposing faces shaped to engage a major portion of the surface of the adjacent vertebrae.

5. A spinal insert of claim 4 wherein said insert a space between said faces defined by a side wall, said side wall containing apertures into said space, said space adapted to be filled with bone growth material.

6. A modular spinal implant adapted to fuse adjacent vertebrae for stabilizing the spine comprising a main body and an interchangeable insert sized to approximate the intervertebral disk space, said main body being elongated and having a longitudinally extending slot, said interchangeable insert having opposing planar faces connected by a continuous peripheral side wall, said planar faces adapted to engage adjacent vertebrae, said planar faces and peripheral side wall sized to approximate the surface of the adjacent vertebrae, said side wall including an integral fin, said side wall and said planar faces defining a space adapted to contain bone growth material, said side wall containing apertures, said fin inserted in said longitudinal slot, and said fin interlocked with said longitudinal slot whereby a proper sized insert may be connected to the main body and bony ingrowth is facilitated.

7. A spinal implant of claim 6 wherein said main body includes an exterior surface, said exterior surface having structure for promoting bony ingrowth.
